(19) United States
(12) Patent Application Publication
(10) Pub. No.: US 2026/0191536 A1
(43) Pub. Date: Jul. 9, 2026

(54) LIGAMENT-BALANCED PIN GUIDE INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Jeremiah M. Lewis, Warsaw, IN (US); Jason M. Chavarria, Warsaw, IN (US)

(21) Appl. No.: 19/417,750

(22) Filed: Dec. 12, 2025

Related U.S. Application Data
(60) Provisional application No. 63/741,615, filed on Jan. 3, 2025.

(51) Int. Cl. A61B 17/15

(52) U.S. Cl. CPC A61B 17/155; A61B 17/157

(57) ABSTRACT

A ligament-balanced pin guide instrument includes a ligament balancer, a mounting block, an extension pin guide, and a flexion pin guide. The mounting block is used to selectively secure the extension pin guide and the flexion pin guide to the ligament balancer. The ligament balancer is configured to be positioned between an unresected femoral condyle of a femur and a resected tibia of a patient so that guide pins can be installed into the femur by selective use of the extension pin guide and the flexion pin guide for locating a distal femoral cutting block and a 4-in-1 cutting block. A surgical instrument kit and methods of surgically preparing a patient's femur and tibia are also disclosed.

This application claims priority to U.S. Provisional Patent Application Ser. No. 63/741,615 which was filed on Jan. 3, 2025, which is hereby incorporated by reference in its entirety.

### TECHNICAL FIELD

The present disclosure relates generally to orthopaedic surgical instruments and, more particularly, to surgical instruments used during an orthopaedic surgical knee procedure.

### BACKGROUND

Joint arthroplasty is a well-known surgical procedure by which a diseased and/or damaged natural joint is replaced by a prosthetic joint. For example, in a total knee arthroplasty surgical procedure, a patient's natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient's knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient's knee.

### SUMMARY

According to one aspect of the disclosure, an orthopaedic surgical instrument assembly for use during an orthopaedic knee replacement procedure includes a ligament balancer, a mounting block, an extension pin guide, and a flexion pin guide. The ligament balancer is configured to be positioned between an unresected femoral condyle of a femur and a resected tibia of a patient. The ligament balancer includes a tibial plate and a femoral plate movable upwardly and downwardly relative to the tibial plate. The mounting block is configured to be removably secured to the ligament balancer. The mounting block has a guide connector. The extension femoral pin guide has a guide connector configured to be selectively secured to the guide connector of the mounting block. The extension femoral pin guide has a pair of pin guide holes configured to locate placement of a distal femoral cutting block on an anterior surface of the femur of the patient. The flexion femoral pin guide has a guide connector configured to be selectively secured to the guide connector of the mounting block. The flexion femoral pin guide has a plurality of pairs of pin guide holes configured to locate placement of a 4-in-1 femoral cutting block on a resected distal surface of the femur of the patient.

In an example, the ligament balancer further includes a block connector. The mounting block includes a block connector configured to be removably secured to the block connector of the ligament balancer.

In an example, the block connector of the ligament balancer includes a connector plate having a pair of mounting bores defined therein, and the block connector of the mounting block includes a pair of mounting pins that are configured to be received into the mounting bores of the connector plate so as to secure the mounting block to the ligament balancer.

In another example, the connector plate is secured to an upper surface of the tibial plate of the ligament balancer.

In another example, the ligament balancer further comprises a drive assembly operable to move the femoral plate upwardly and downwardly relative to the tibial plate, and the drive assembly of the ligament balancer includes a drive screw, and the drive screw extends through the connector plate of the block connector of the ligament balancer between the pair of mounting bores.

The extension femoral pin guide may include a guide plate and a grip flange extending anteriorly away from the guide plate, with the pair of guide pin holes being defined in the guide plate. The guide connector of the mounting block has an anteroposteriorly-extending slot formed therein, and the guide connector of the extension femoral pin guide has a pair of anteroposteriorly-extending rails formed in the grip flange. The rails are configured to be received into the slot formed in the mounting block.

The flexion femoral pin guide may include a guide plate and a grip flange extending anteriorly away from the guide plate, with the plurality of pairs of guide pin holes being defined in the guide plate. The guide connector of the mounting block has an anteroposteriorly-extending slot formed therein, and the guide connector of the flexion femoral pin guide has a pair of anteroposteriorly-extending rails formed in the grip flange. The rails are configured to be received into the slot of the mounting block.

In an example, the ligament balancer further comprises a drive assembly operable to move the femoral plate upwardly and downwardly relative to the tibial plate, and the drive assembly of the ligament balancer includes a drive screw having a cam block secured thereto. Rotation of the drive screw in a first rotational direction causes the cam block to be urged in a first linear direction so as to move the femoral plate upwardly away from the tibial plate, whereas rotation of the drive screw in a second, opposite rotational direction causes the cam block to be urged in a second, opposite linear direction so as to move the femoral plate downwardly toward the tibial plate.

The cam block may have an upwardly-facing ramp surface formed therein, with a lower surface of the femoral plate having a downwardly-facing ramp surface formed therein. Rotation of the drive screw in the first rotational direction urges the ramp surface of the cam block inwardly into the ramp surface of the femoral plate so as to move the femoral plate upwardly away from the tibial plate, whereas rotation of the drive screw in the second rotational direction urges the ramp surface of the cam block outwardly away from the ramp surface of the femoral plate so as to move the femoral plate downwardly toward the tibial plate.

In an example, the tibial plate has an anteroposteriorly-extending center line, and a longitudinal axis of the drive screw is colinear with the center line of the tibial plate.

In an example, the drive screw includes a socket defined in a proximal end thereof and a threaded shaft extending distally away from the socket.

According to another aspect, a method of surgically preparing a patient's femur and tibia during an orthopaedic surgical knee procedure includes resecting a proximal end of the patient's tibia. The method also includes positioning the patient's femur and tibia in extension. With the patient's femur and tibia positioned in extension, a ligament balancer is installed between the resected proximal end of the patient's tibia and an unresected pair of distal femoral condyles of the patient's femur. With the patient's femur and tibia positioned in extension, an extension pin guide is secured to the installed ligament balancer. Thereafter, a pair of guide pins is installed into an anterior surface of the patient's femur with the extension pin guide. The extension pin guide is then removed and a distal femoral cutting block is installed onto the pair of guide pins installed on the anterior surface of the patient's femur. A distal femoral resection is performed on the patient's femur with the installed distal femoral cutting block so as to produce a planar resected distal femoral surface. The method includes positioning the patient's femur and tibia in flexion. With the patient's femur and tibia positioned in flexion, the ligament balancer is installed between the resected proximal end of the patient's tibia and an unresected pair of posterior femoral condyles of the patient's femur. With the patient's femur and tibia positioned in flexion, a flexion pin guide is secured to the installed ligament balancer. A pair of guide pins is then installed into the resected distal surface of the patient's femur with the flexion pin guide. The flexion pin guide is then removed and a 4-in-1 femoral cutting block is installed onto the pair of guide pins installed in the resected distal surface of the patient's femur. A number of additional femoral resections are performed on the patient's femur with the installed 4-in-1 femoral cutting block.

In an example, with the patient's femur and tibia positioned in extension, the installed ligament balancer is operated to tension a number of knee ligaments of the patient prior to installation of the extension pin guide.

In an example, a drive assembly of the ligament balancer is operated to move a femoral plate of the ligament balancer away from a tibial plate of the ligament balancer.

Such operation of the drive assembly of the ligament balancer may include urging a cam block into the femoral plate to move the femoral plate of the ligament balancer away from the tibial plate of the ligament balancer.

In an example, with the patient's femur and tibia positioned in flexion, the installed ligament balancer is operated to tension a number of knee ligaments of the patient prior to installation of the extension pin guide.

In an example, a drive assembly of the ligament balancer is operated to move a femoral plate of the ligament balancer away from a tibial plate of the ligament balancer.

Such operation of the drive assembly of the ligament balancer may include urging a cam block into the femoral plate to move the femoral plate of the ligament balancer away from the tibial plate of the ligament balancer.

The method may also include, with the patient's femur and tibia positioned in extension, securing a mounting block to the installed ligament balancer. The method may also include securing the extension pin guide to the mounting block.

The method may also include, with the patient's femur and tibia positioned in flexion, securing a mounting block to the installed ligament balancer. The method may also include securing the flexion pin guide to the mounting block.

According to another aspect, a method of surgically preparing a patient's femur and tibia during an orthopaedic surgical knee procedure includes resecting a proximal end of the patient's tibia and performing a distal femoral resection on the patient's femur so as to produce a planar resected distal femoral surface. The method includes positioning the patient's femur and tibia in flexion. With the patient's femur and tibia positioned in flexion, the ligament balancer is installed between the resected proximal end of the patient's tibia and an unresected pair of posterior femoral condyles of the patient's femur. With the patient's femur and tibia positioned in flexion, a flexion pin guide is secured to the installed ligament balancer. A pair of guide pins is then installed into the resected distal surface of the patient's femur with the flexion pin guide. The flexion pin guide is then removed and a 4-in-1 femoral cutting block is installed onto the pair of guide pins installed in the resected distal surface of the patient's femur. A number of additional femoral resections are performed on the patient's femur with the installed 4-in-1 femoral cutting block.

In an example, with the patient's femur and tibia positioned in flexion, the installed ligament balancer is operated to tension a number of knee ligaments of the patient prior to installation of the flexion pin guide.

In an example, a drive assembly of the ligament balancer is operated to move a femoral plate of the ligament balancer away from a tibial plate of the ligament balancer.

Such operation of the drive assembly of the ligament balancer may include urging a cam block into the femoral plate to move the femoral plate of the ligament balancer away from the tibial plate of the ligament balancer.

The method may also include, with the patient's femur and tibia positioned in flexion, securing a mounting block to the installed ligament balancer. The method may also include securing the flexion pin guide to the mounting block.

According to another aspect, an orthopaedic surgical instrument kit for use during an orthopaedic knee replacement procedure includes a distal femoral cutting block configured to guide a surgical saw blade in the performance of a distal femoral resection. The distal femoral cutting block has a plurality of pin holes. The kit also includes a 4-in-1 femoral cutting block configured to guide a surgical saw blade in the performance of (i) an anterior femoral resection, (ii) a posterior femoral resection, and (ii) a pair of chamfer femoral resections. The 4-in-one femoral cutting block having a plurality of pin holes. The kit also includes a ligament balancer that is configured to be positioned between an unresected femoral condyle of a femur and a resected tibia of a patient. The ligament balancer includes a tibial plate, a femoral plate movable upwardly and downwardly relative to the tibial plate, and a drive assembly operable to move the femoral plate upwardly and downwardly relative to the tibial plate. The kit also includes a mounting block that is configured to be removably secured to the ligament balancer. The mounting block has a guide connector. The kit further includes an extension femoral pin guide having a guide connector configured to be selectively secured to the guide connector of the mounting block. The extension femoral pin guide has a plurality of pin guide holes that are arranged in a hole pattern and spacing arrangement that matches a hole pattern and spacing arrangement in which the pin holes of the distal femoral cutting block are arranged. The kit also includes a flexion femoral pin guide having a guide connector configured to be selectively secured to the guide connector of the mounting block. The flexion femoral pin guide has a plurality of pin guide holes that are arranged in a hole pattern and spacing arrangement that matches a hole pattern and spacing arrangement in which the pin holes of the 4-in-1 femoral cutting block are arranged.

In an example, the ligament balancer further includes a block connector. The mounting block includes a block connector configured to be removably secured to the block connector of the ligament balancer.

In an example, the block connector of the ligament balancer includes a connector plate having a pair of mounting bores defined therein, and the block connector of the mounting block includes a pair of mounting pins that are configured to be received into the mounting bores of the connector plate so as to secure the mounting block to the ligament balancer.

In another example, the connector plate is secured to an upper surface of the tibial plate of the ligament balancer.

The extension femoral pin guide may include a guide plate and a grip flange extending anteriorly away from the guide plate, with the pair of guide pin holes being defined in the guide plate. The guide connector of the mounting block has an anteroposteriorly-extending slot formed therein, and the guide connector of the extension femoral pin guide has a pair of anteroposteriorly-extending rails formed in the grip flange. The rails are configured to be received into the slot formed in the mounting block.

The flexion femoral pin guide may include a guide plate and a grip flange extending anteriorly away from the guide plate, with the plurality of pairs of guide pin holes being defined in the guide plate. The guide connector of the mounting block has an anteroposteriorly-extending slot formed therein, and the guide connector of the flexion femoral pin guide has a pair of anteroposteriorly-extending rails formed in the grip flange. The rails are configured to be received into the slot of the mounting block.

In an example, the drive assembly of the ligament balancer includes a drive screw having a cam block secured thereto. Rotation of the drive screw in a first rotational direction causes the cam block to be urged in a first linear direction so as to move the femoral plate upwardly away from the tibial plate, whereas rotation of the drive screw in a second, opposite rotational direction causes the cam block to be urged in a second, opposite linear direction so as to move the femoral plate downwardly toward the tibial plate.

The cam block may have an upwardly-facing ramp surface formed therein, with a lower surface of the femoral plate having a downwardly-facing ramp surface formed therein. Rotation of the drive screw in the first rotational direction urges the ramp surface of the cam block inwardly into the ramp surface of the femoral plate so as to move the femoral plate upwardly away from the tibial plate, whereas rotation of the drive screw in the second rotational direction urges the ramp surface of the cam block outwardly away from the ramp surface of the femoral plate so as to move the femoral plate downwardly toward the tibial plate.

In an example, the tibial plate has an anteroposteriorly-extending center line, and a longitudinal axis of the drive screw is colinear with the center line of the tibial plate.

### BRIEF DESCRIPTION OF THE DRAWINGS

The detailed description particularly refers to the following figures, in which:

FIG. 1 is an exploded perspective view of a ligament-balanced pin guide instrument for use in the surgical preparation of a patient's femur during performance of an orthopaedic knee procedure;

FIG. 2 is an exploded perspective view showing the extension pin guide being coupled to the assembled mounting block and ligament balancer of the ligament-balanced pin guide instrument of FIG. 1;

FIG. 3 is a view similar to FIG. 2, but showing the flexion pin guide being coupled to the assembled mounting block and ligament balancer;

FIG. 4 is a front perspective view of the ligament balancer of the ligament-balanced pin guide instrument of FIG. 1;

FIG. 5 is a view similar to FIG. 4, but showing the drive assembly being operated to move the femoral plate of the ligament balancer away from the tibial plate;

FIG. 6 is a front view of the ligament balancer of FIG. 4 showing the drive assembly being operated to move the femoral plate of the ligament balancer away from the tibial plate;

FIG. 7 is a side perspective view of the ligament balancer of the ligament-balanced pin guide instrument of FIG. 1;

FIG. 8 is a side view of the ligament balancer of the ligament-balanced pin guide instrument of FIG. 1;

FIG. 9 is a fragmentary cross sectional view taken along the line 9-9 of FIG. 4, as viewed in the direction of the arrows;

FIG. 10 is a front view of the ligament balancer of the ligament-balanced pin guide instrument installed in the patient's knee, note the patient's knee is positioned in extension in FIG. 10;

FIG. 11 is a view similar to FIG. 10, but showing the extension pin guide secured to the ligament balancer via use of the mounting block;

FIG. 12 is a perspective view showing a distal cutting block installed on the distal end of the patient's femur and being used during performance of a distal resection of the patient's femur;

FIG. 13 is a front view of the ligament balancer of the ligament-balanced pin guide instrument installed in the patient's knee, note the patient's knee is positioned in flexion in FIG. 13;

FIG. 14 is a view similar to FIG. 13, but showing the flexion pin guide secured to the ligament balancer via use of the mounting block; and

FIG. 15 is a perspective view showing a 4-in-1 cutting block installed on the distal end of the patient's femur and being used during performance of an anterior resection of the patient's femur.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

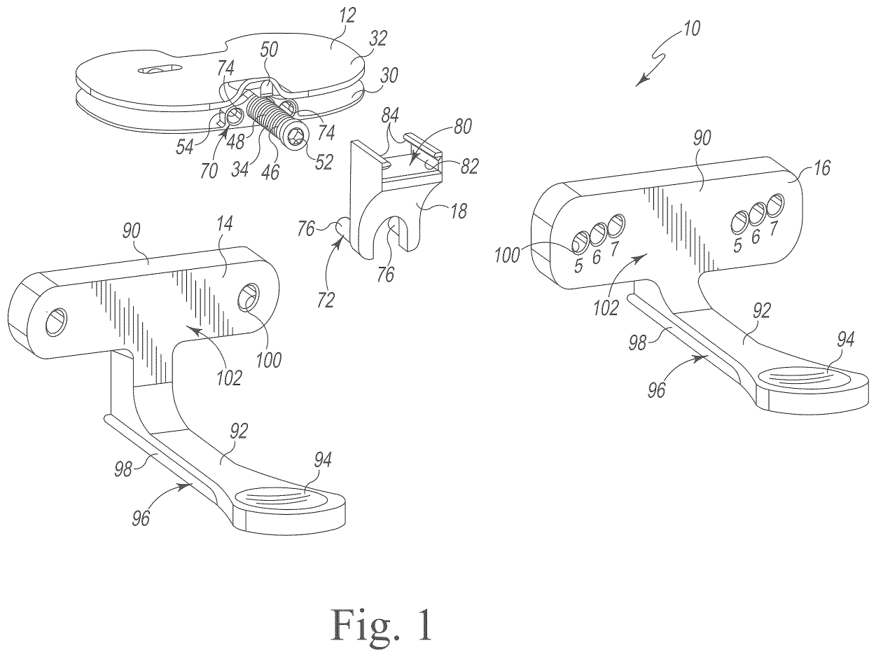

Referring to FIGS. 1-3, an orthopaedic surgical instrument 10 in the form of a ligament-balanced pin guide instrument-for use in the surgical preparation of a patient's femur during performance of an orthopaedic knee procedure is shown. As will be described below in more detail, the ligament-balanced pin guide instrument 10 may be used by a surgeon to install a distal femoral cutting block 112 and an A/P chamfer block 142, commonly known as a 4-in-1 cutting block, on the distal end 126 of the patient's femur 120. The ligament-balanced pin guide instrument 10 may be used by a surgeon in a tibia-first balanced surgical technique. In such a technique, the surgeon resects the proximal end 140 of the patient's tibia 130 prior to performing the resections on the patient's femur 120. The cutting angles of the femoral resections are determined using a balanced approach that sets the resection planes based on ligament tension.

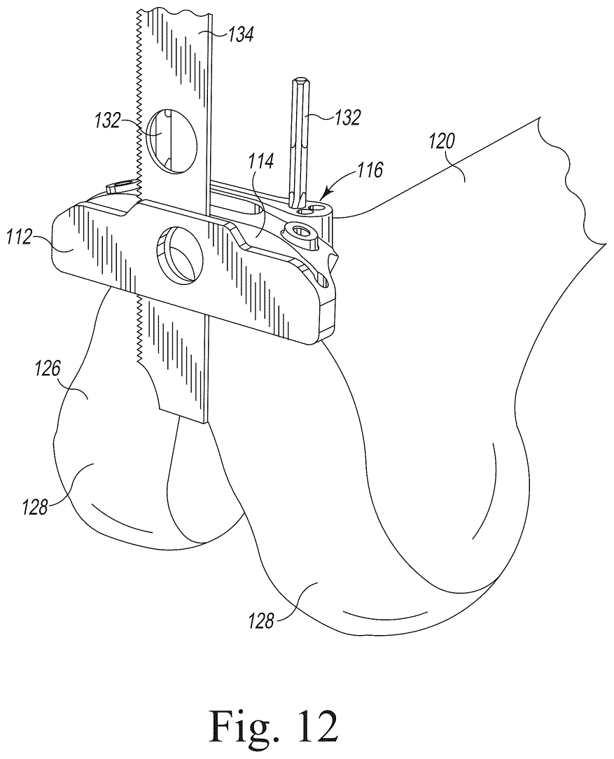

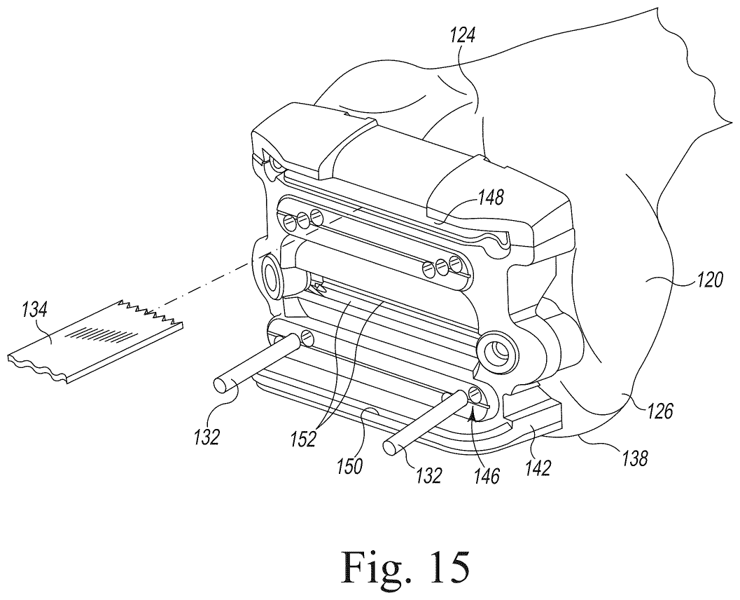

The ligament-balanced pin guide instrument 10 includes a ligament balancer 12, an extension pin guide 14, a flexion pin guide 16, and a mounting block 18 for selectively securing the pin guides 14, 16 to the ligament balancer 12. As will be described in more detail below, the ligament balancer 12 may be inserted into the knee joint gap after performance of a proximal cut on the patient's tibia 130 to balance the extension gap and the flexion gap. After such balancing of the extension gap, the extension pin guide 14 may be installed on the ligament balancer 12 by use of the mounting block 18 such that a pair of surgical pins 132 can be installed on an anterior surface of the patient's femur 120 with the extension pin guide 14. Thereafter, a distal femoral cutting block 112 may be installed on the surgical pins 132 to guide the surgeon in the performance of a distal femoral resection, as shown in FIG. 12. The surgeon may then place the patient's knee in flexion and locate a pair of surgical pins 132 on the resected distal surface of the patient's knee by securing the flexion pin guide 16 to the installed ligament balancer 12. A 4-in-1 cutting block 142 may then be installed on the surgical pins 132 to guide the surgeon in the performance of an anterior resection, a posterior resection, and a pair of chamfer resections on the patient's femur 120, as shown in FIG. 15.

As can be seen in FIG. 1, the ligament balancer 12 includes a tibial plate 30 and a femoral plate 32 that is movable upwardly and downwardly relative to the tibial plate 30. The ligament balancer 12 also includes a drive assembly 34 that is operable to move the femoral plate 32 upwardly and downwardly relative to the tibial plate 30. As can be seen in FIGS. 6-8, a connecting arm 36 couples the two plates 30, 32 to one another. More specifically, one end 38 of the connecting arm 36 is pivotally coupled to the tibial plate 30 with a pivot pin 40, with the opposite end 42 of the connecting arm 36 being pivotally coupled to the femoral plate 32 with a pivot pin 44. In such a way, the femoral plate 32 is permitted to not only move upwardly and downwardly relative to the tibial plate 30, but also pivot relative to the tibial plate 30.

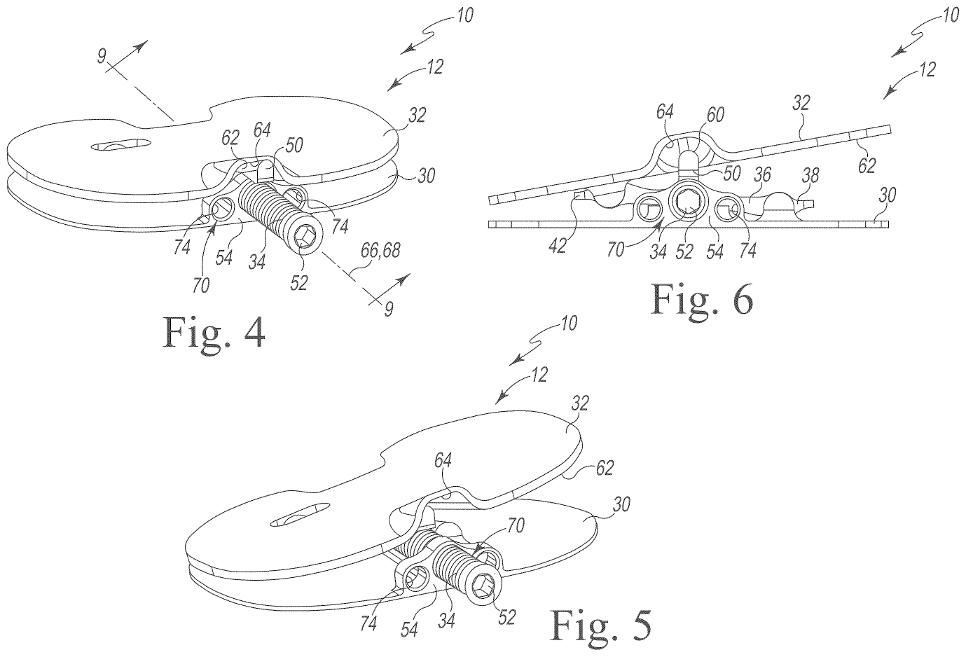

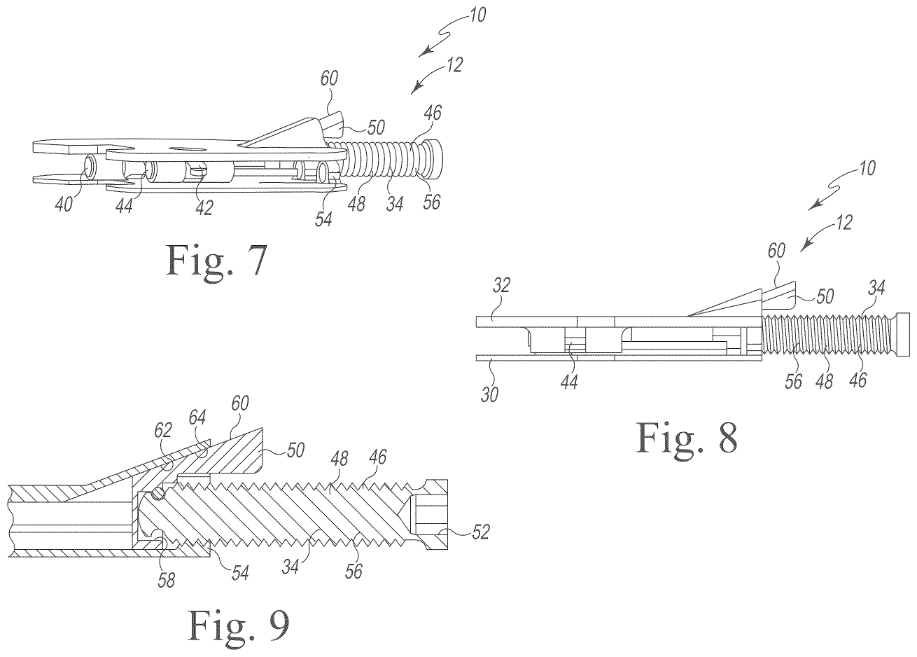

The drive assembly 34 is operable to move the femoral plate 32 upwardly and downwardly relative to the tibial plate 30 so as to tension the ligaments of the patient's knee when the ligament balancer 12 is positioned in the extension gap or flexion gap. In the example described herein, the drive assembly 34 is embodied as a threaded drive assembly 46 that is operatively coupled to the femoral plate 32 to move the femoral plate 32 between its raised position and its lowered position. Specifically, as best seen in FIGS. 7-9, the threaded drive assembly 46 includes a drive screw 48 having a cam block 50 captured on its distal end. The drive screw 48 is rotatably coupled to a connector plate 54 formed in or otherwise secured to the tibial plate 30. As can be seen best in FIG. 4, the longitudinal axis 66 of the drive screw 48 is colinear with the anteroposteriorly-extending center line 68 of the tibial plate 30. The drive screw 48 includes a socket 52 defined in its proximal end. A threaded shaft 56 extends distally away from the socket 52. The cam block 50 has a flat lower surface so as to be slidable or otherwise movable along the upper surface of tibial plate 30 in a direction toward, and a direction away from, the center of the tibial plate 30. The cam block 50 has a non-threaded cavity 58 formed therein. The non-threaded distal end of the threaded shaft 56 of the drive screw 48 is captured in the non-threaded cavity 58 of the cam block 50, as shown in FIG. 9. The distal end of the threaded shaft 56 freely rotates within the cam block's non-threaded cavity 58. Thus, rotation of the threaded shaft 56 of the drive screw 48 causes linear movement of the cam block 50 along the upper surface of tibial plate 30, but does not create corresponding rotation of the cam block 50.

As can be seen best in FIGS. 7-9, the cam block 50 has an upwardly-facing ramp surface 60 formed therein. A lower surface 62 of the femoral plate 32 has a correspondingly-shaped downwardly-facing ramp surface 64 formed therein. As can be seen in FIGS. 4-6, the socket 52 of the drive screw 48 is configured as a hex socket configured to receive a manual or powered hex driver (not shown). When a surgeon or other personnel rotates the drive screw 48, the drive screw's threaded shaft 56 is likewise rotated thereby causing linear movement of the cam block 50. Rotation in one direction (e.g., clockwise) moves the cam block 50 and hence its ramp surface 60 in the direction toward the center of the femoral plate 32 thereby urging the femoral plate 32 upwardly as the cam block's ramp surface 60 moves posteriorly into contact with the femoral plate's ramp surface 64. Rotation in the opposite direction (e.g., counterclockwise) moves the cam block 50 and hence its ramp surface 60 in the direction away from the center of the femoral plate 32 thereby allowing the femoral plate 32 to move downwardly as the cam block's ramp surface 60 moves anteriorly along the femoral plate's ramp surface 64.

As can be seen in FIGS. 1 and 4-6, the ligament balancer 12 also includes a block connector 70 that mates with a block connector 72 of the mounting block 18 to removably secure the mounting block 18 to the ligament balancer 12. In the example described herein, the ligament balancer's block connector 70 is embodied as a pair of mounting bores 74 formed in the connector plate 54 of the tibial plate 30. In the example described herein, the mounting block's block connector 72 is embodied as a pair of the mounting pins 76 that are configured to be received into the mounting bores 74 of the connector plate 54 so as to secure the mounting block 18 to the ligament balancer 12, as shown in FIGS. 1-3.

The mounting block 18 has a guide connector 80 formed in its superior end. In the example described herein, the guide connector 80 is embodied as an anteroposteriorly-extending slot 82 having a pair of anteroposteriorly-extending capture rails 84, one of which is positioned on the medial and lateral sides thereof. The extension pin guide 14 and the flexion pin guide 16 each include a guide plate 90 and a grip flange 92 extending anteriorly away from the guide plate 90. The proximal end of the grip flange 92 has a finger grip 94 formed therein. The finger grip 94 may be pinched or otherwise gripped by the surgeon during installation or removal of the pin guide 14, 16. The pin guides 14, 16 have a guide connector 96 that mates with the guide connector 80 of the mounting block 18 to selectively secure the pin guides 14, 16 to the mounting block 18. In the example described herein, the guide connector 96 of the pin guides 14, 16 is embodied as a pair of anteroposteriorly-extending rails 98, one of which formed on the medial and lateral sides of the grip flange 92. The grip's rails 98 are configured to be received into the slot 82 formed in the mounting block 18. The grip's rails 98 are captured in the slot 82 by the block's capture rails 84.

The guide plate 90 of the extension pin guide 14 and the flexion pin guide 16 have a number of guide holes 100 formed therein. The guide holes 100 are configured to guide the placement and installation of surgical guide pins 132. The guide holes 100 are arranged in a hole pattern and spacing arrangement 102 that coincides with a cutting block to be used at a particular step in the surgical preparation of the patient's femur 120. For example, the guide holes 100 of the extension pin guide 14 are arranged in a hole pattern and spacing arrangement 102 that matches the hole pattern and spacing arrangement 116 on a distal femoral cutting block 112. Similarly, the guide holes 100 of the flexion pin guide 16 are arranged in hole pattern and spacing arrangement 102 that matches the hole pattern and spacing arrangement 146 on a 4-in-1 femoral cutting block 142. As can be seen in, for example, FIGS. 1-3, 11, and 14, the holes of a given pair of the guide holes 100 (e.g., the lone pair of holes 100 of the extension pin guide 14 or the size #5 holes 100 of the flexion pin guide 16) are oriented generally parallel to the tibial plate 30. In other words, an imaginary line connecting the centers of a given pair of the guide holes 100 extends parallel to the lower surface (and its opposite upper surface) of the tibial plate 30 of the ligament balancer 12. It should be appreciated that other arrangements are also contemplated for use in the design of the pin guides 14, 16. In particular, non-parallel arrangements of the guide holes 100 may be used. For example, a trapezoidal joint gap could be created by using a pin guide having +1 mm or +2 mm pin hole arrangement. Such a pin guide could be used to open up the lateral gap by 1 mm or 2 mm, for example.

In operation, the surgeon may utilize the ligament-balanced pin guide instrument 10 during performance of an orthopaedic knee procedure to prepare the distal end 126 of the patient's femur 120 to receive a prosthetic femoral component. In doing so, the surgeon may utilize the ligament-balanced pin guide instrument 10 as part of a tibia-first ligament balanced approach to perform the resections on the patient's femur 120.

During such an orthopaedic surgical procedure, the surgeon first performs a tibial resection on the patient's tibia 130 thereby producing a planar resected surface 136 on the proximal end 140 of the patient's tibia 130. The surgeon may utilize any number of different techniques to perform the tibial resection of the patient's tibia 130. In the example described herein, the surgeon may perform the tibial resection of the patient's tibia 130 by utilizing the techniques taught in copending U.S. Patent Application Ser. No. 63/741,632 entitled “TIBIAL ALIGNMENT JIG INSTRUMENT AND METHOD OF USING THE SAME IN AN ORTHOPAEDIC SURGICAL KNEE PROCEDURE” (Attorney Docket No. 265280-417981, DEP7190USPSP1), which was filed on Jan. 3, 2025, is assigned to the same assignee as the present application, and hereby incorporated by reference in its entirety.

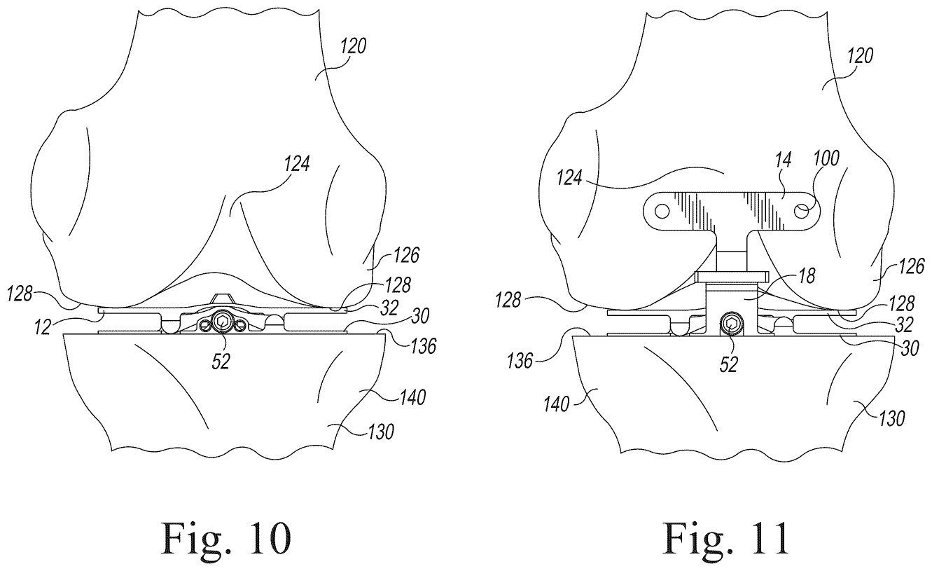

Subsequent to performing the tibial resection on the patient's tibia 130, the surgeon orientates the patient's femur 120 and tibia 130 such that the patient's knee is positioned in extension, as shown in FIGS. 10 and 11. With the patient's knee positioned in extension, the surgeon installs the ligament-balanced pin guide instrument 10. To do so, the ligament balancer 12 is inserted into the joint gap between the patient's unresected femur 120 and the patient's resected tibia 130. During such installation, as shown in FIG. 10, the flat upper surface of the femoral plate 32 contacts the unresected distal femoral condyles 128 of the distal end 126 of the patient's femur 120 and the flat lower surface of the tibial plate 30 contacts the planar resected surface 136 of the proximal end 140 of the patient's tibia 130.

The surgeon may then use the installed ligament balancer 12 of the ligament-balanced pin guide instrument 10 to perform a ligament balance of the patient's knee. To do so, the surgeon inserts a manual or powered hex driver (not shown) into the socket 52 of the drive screw 48 and rotates the socket 52 and hence the drive screw 48 in a direction (e.g., clockwise) to move the cam block 50 and hence its ramp surface 60 in the direction toward the center of the femoral plate 32 thereby urging the femoral plate 32 upwardly as the cam block's ramp surface 60 moves posteriorly into contact with the femoral plate's ramp surface 64. As the femoral plate 32 is moved upwardly away from the tibial plate 30, it may pivot relative to one or both ends of the connecting arm 36. Such upward movement of the femoral plate 32 increases ligament tension of the patient's knee ligaments. The surgeon does an initial assessment of the ligament tension on the patient's knee and then, if need be, further upwardly advances the femoral plate 32. Based on the initial assessment, if need be, the surgeon may rotate the socket 52 and hence the drive screw 48 in an opposite direction (e.g., counterclockwise) to move the cam block 50 and hence the ramp surface 60 in the direction away from the center of the femoral plate 32 thereby allowing the femoral plate 32 to move downwardly as the cam block's ramp surface 60 moves anteriorly along the femoral plate's ramp surface 64. Doing so lessens ligament tension of the patient's knee ligaments. The surgeon may continue to assess the balance of the patient's knee by operating the ligament balancer 12 until the surgeon is satisfied with the balance of the patient's knee.

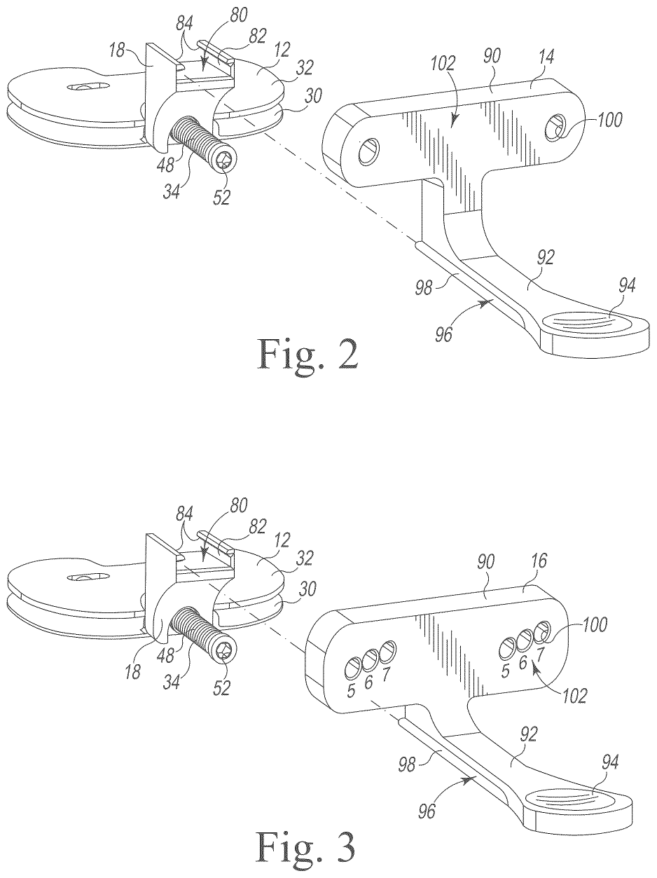

Once the surgeon is satisfied with the balance of the patient's knee, the surgeon may then install the extension pin guide 14. To do so, the surgeon first installs the mounting block 18 to the ligament balancer 12 by inserting the mounting block's mounting pins 76 into the mounting bores 74 of the ligament balancer's connector plate 54, as shown in FIGS. 2 and 11. With the mounting block 18 installed, the surgeon may then pinch or otherwise grip the finger grip 94 of the extension pin guide 14 and insert the opposite, distal end of the grip flange 92 into the slot 82 formed in the mounting block 18 such that the rails 98 formed on the medial and lateral sides of the grip flange 92 are captured in the slot 82 by the block's capture rails 84. The surgeon slides the grip flange 92 posteriorly until the posterior side of the guide plate 90 of the extension pin guide 14 abuts the anterior surface 124 of the patient's femur 120, as shown in FIG. 11.

Once the surgeon has so positioned the extension pin guide 14, the surgeon may install a pair of guide pins 132 through the pin guide's guide holes 100. The surgeon may then remove the extension pin guide 14 and install the distal femoral cutting block 112 on the installed guide pins 132, as shown in FIG. 12. With the distal femoral cutting block 112 installed on the distal end 126 of the patient's femur 120, the surgeon may then use the distal femoral cutting block 112 to perform a distal resection of the distal end 126 of the patient's femur 120. Specifically, the surgeon may advance the bone saw blade 134 of a surgical saw through the distal cutting slot 114 to engage the patient's femur 120 and operate the surgical saw to surgically form a planar resected surface 122 of the distal end 126 of the patient's femur 120.

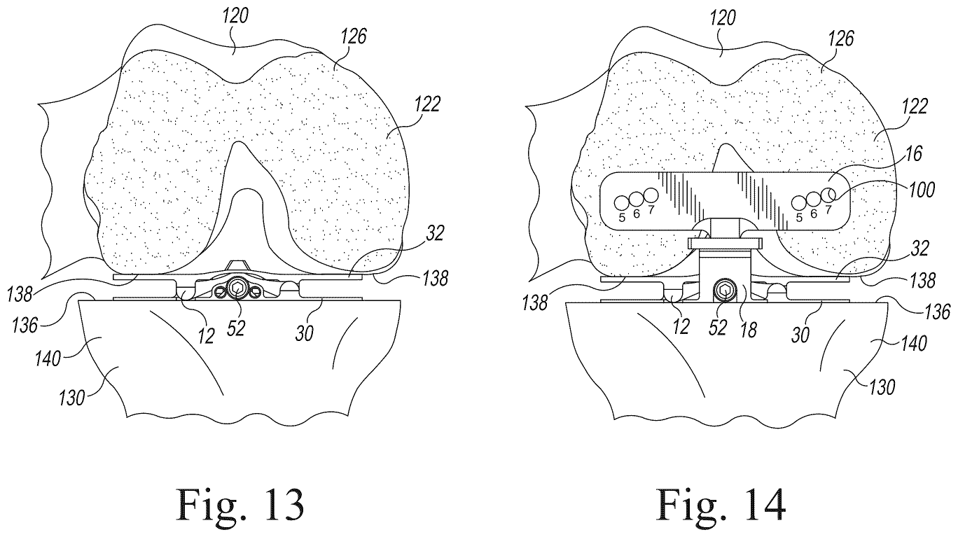

Subsequent to performing the distal femoral resection on the patient's femur 120, the surgeon orientates the patient's femur 120 and tibia 130 such that the patient's knee is positioned in flexion, as shown in FIGS. 13 and 14. With the patient's knee positioned in flexion, the surgeon reinstalls the ligament-balanced pin guide instrument 10. To do so, the ligament balancer 12 is inserted into the joint gap between the unresected posterior femoral condyles 138 of the patient's femur 120 and the patient's resected tibia 130. During such installation, as shown in FIG. 13, the flat upper surface of the femoral plate 32 contacts the unresected posterior femoral condyles 138 of the distal end 126 of the patient's femur 120 and the flat lower surface of the tibial plate 30 contacts the planar resected surface 136 of the proximal end 140 of the patient's tibia 130.

The surgeon may then use the installed ligament balancer 12 of the ligament-balanced pin guide instrument 10 to perform a ligament balance of the patient's knee. To do so, the surgeon inserts a manual or powered hex driver (not shown) into the socket 52 of the drive screw 48 and rotates the socket 52 and hence the drive screw 48 in the direction (e.g., clockwise) to move the cam block 50 and hence its ramp surface 60 in the direction toward the center of the femoral plate 32 thereby urging the femoral plate 32 upwardly as the cam block's ramp surface 60 moves posteriorly into contact with the femoral plate's ramp surface 64. As the femoral plate 32 is moved upwardly away from the tibial plate 30, it may pivot relative to one or both ends of the connecting arm 36. Such upward movement of the femoral plate 32 increases ligament tension of the patient's knee ligaments. The surgeon does an initial assessment of the ligament tension on the patient's knee and then, if need be, further upwardly advances the femoral plate 32. Based on the initial assessment, if need be, the surgeon may rotate the socket 52 and hence the drive screw 48 in the opposite direction (e.g., counterclockwise) to move the cam block 50 and hence its ramp surface 60 in the direction away from the center of the femoral plate 32 thereby allowing the femoral plate 32 to move downwardly as the cam block's ramp surface 60 moves anteriorly along the femoral plate's ramp surface 64. Doing so lessens ligament tension of the patient's knee ligaments. The surgeon may continue to assess the balance of the patient's knee by operating the ligament balancer 12 until the surgeon is satisfied with the balance of the patient's knee.

Once the surgeon is satisfied with the balance of the patient's knee position in flexion, the surgeon may then install the flexion pin guide 16. To do so, the surgeon first installs the mounting block 18 to the ligament balancer 12 by inserting the mounting block's mounting pins 76 into the mounting bores 74 of the ligament balancer's connector plate 54, as shown in FIGS. 3 and 14. With the mounting block 18 installed, the surgeon may then pinch or otherwise grip the finger grip 94 of the flexion pin guide 16 and insert the opposite, distal end of the grip flange 92 into the slot 82 formed in the mounting block 18 such that the rails 98 formed on the medial and lateral sides of the grip flange 92 are captured in the slot 82 by the block's capture rails 84. The surgeon slides the grip flange 92 posteriorly until the posterior side of the guide plate 90 of the flexion pin guide 16 abuts the resected distal surface 122 of the patient's femur 120, as shown in FIG. 14.

Once the surgeon has so positioned the flexion pin guide 16, the surgeon may install a pair of guide pins 132 through the pin guide's guide holes 100. The surgeon may then remove the flexion pin guide 16 and install the 4-in-1 femoral cutting block 142 on the installed guide pins 132, as shown in FIG. 15. With the 4-in-1 femoral cutting block 142 installed on the distal end 126 of the patient's femur 120, the surgeon may then use the 4-in-1 cutting block 142 to perform a number of additional resections of the distal end 126 of the patient's femur 120. Specifically, the surgeon may advance the bone saw blade 134 of the surgical saw through the anterior cutting slot 148 to engage the patient's femur 120 and operate the surgical saw to surgically resect the anterior surface 124 of the patient's femur 120. The surgeon may similarly use the posterior cutting slot 150 to resect the posterior femoral condyles 138 of the patient's femur 120. The surgeon may also use the chamfer cutting slots 152 to make a pair of chamfer cuts on the patient's femur 120.

Once the anterior cut, posterior cut, and both chamfer cuts have been made, the surgeon removes the 4-in-1 cutting block 142 from the patient's femur 120 and performs the remaining steps of the surgical procedure including installation of a prosthetic femoral implant component onto the resected femur 120 and a prosthetic tibial implant component onto the resected tibial surface.

It should be appreciated that although the flexion pin guide 16 is described herein as being used after the distal femoral resection was performed by use of the extension pin guide 14, other surgical workflows are also contemplated for use by a surgeon. For example, the flexion pin guide 16 and ligament balancer 12 may be used as described herein to install the 4-in-1 cutting block 142 after the distal femoral resection was performed as taught in copending U.S. Patent Application Ser. No. 63/741,619 entitled “DISTAL FEMORAL JIG INSTRUMENT AND METHOD OF USING THE SAME IN AN ORTHOPAEDIC SURGICAL KNEE PROCEDURE” (Attorney Docket No. 265280-417983, DEP7189USPSP1), which was filed on Jan. 3, 2025, is assigned to the same assignee as the present application, and hereby incorporated by reference in its entirety.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument assembly for use during an orthopaedic knee replacement procedure, comprising:
a ligament balancer configured to be positioned between an unresected femoral condyle of a femur and a resected tibia of a patient, the ligament balancer having (i) a tibial plate and (ii) a femoral plate movable upwardly and downwardly relative to the tibial plate,
a mounting block configured to be removably secured to the ligament balancer, the mounting block having a guide connector,
an extension femoral pin guide having a guide connector configured to be selectively secured to the guide connector of the mounting block, the extension femoral pin guide having a pair of pin guide holes configured to locate placement of a distal femoral cutting block on an anterior surface of the femur of the patient, and
a flexion femoral pin guide having a guide connector configured to be selectively secured to the guide connector of the mounting block, the flexion femoral pin guide having a plurality of pairs of pin guide holes configured to locate placement of a 4-in-1 femoral cutting block on a resected distal surface of the femur of the patient.

2. The orthopaedic surgical instrument assembly of claim 1, wherein:
the ligament balancer further comprises a block connector, and
the mounting block further comprises a block connector configured to be removably secured to the block connector of the ligament balancer.

3. The orthopaedic surgical instrument assembly of claim 2, wherein:
the block connector of the ligament balancer includes a connector plate having a pair of mounting bores defined therein, and
the block connector of the mounting block includes a pair of mounting pins that are configured to be received into the mounting bores of the connector plate so as to secure the mounting block to the ligament balancer.

4. The orthopaedic surgical instrument assembly of claim 3, wherein the connector plate is secured to an upper surface of the tibial plate of the ligament balancer.

5. The orthopaedic surgical instrument assembly of claim 3, wherein the ligament balancer further comprises a drive assembly operable to move the femoral plate upwardly and downwardly relative to the tibial plate, and wherein the drive assembly of the ligament balancer includes a drive screw, and the drive screw extends through the connector plate of the block connector of the ligament balancer between the pair of mounting bores.

6. The orthopaedic surgical instrument assembly of claim 1, wherein:
the extension femoral pin guide includes a guide plate and a grip flange extending anteriorly away from the guide plate,
the pair of guide pin holes are defined in the guide plate,
the guide connector of the mounting block has an anteroposteriorly-extending slot formed therein, and
the guide connector of the extension femoral pin guide has a pair of anteroposteriorly-extending rails formed in the grip flange, the rails are configured to be received into the slot formed in the mounting block.

7. The orthopaedic surgical instrument assembly of claim 1, wherein:
the flexion femoral pin guide includes a guide plate and a grip flange extending anteriorly away from the guide plate,
the plurality of pairs of guide pin holes are defined in the guide plate,
the guide connector of the mounting block has an anteroposteriorly-extending slot formed therein, and
the guide connector of the flexion femoral pin guide has a pair of anteroposteriorly-extending rails formed in the grip flange, the rails are configured to be received into the slot of the mounting block.

8. The orthopaedic surgical instrument assembly of claim 1, wherein:
the ligament balancer further comprises a drive assembly operable to move the femoral plate upwardly and downwardly relative to the tibial plate,
the drive assembly of the ligament balancer includes a drive screw having a cam block secured thereto,
rotation of the drive screw in a first rotational direction causes the cam block to be urged in a first linear direction so as to move the femoral plate upwardly away from the tibial plate, and
rotation of the drive screw in a second, opposite rotational direction causes the cam block to be urged in a second, opposite linear direction so as to move the femoral plate downwardly toward the tibial plate.

9. The orthopaedic surgical instrument assembly of claim 8, wherein:
the cam block has an upwardly-facing ramp surface formed therein,
a lower surface of the femoral plate has a downwardly-facing ramp surface formed therein,
rotation of the drive screw in the first rotational direction urges the ramp surface of the cam block inwardly into the ramp surface of the femoral plate so as to move the femoral plate upwardly away from the tibial plate, and
rotation of the drive screw in the second rotational direction urges the ramp surface of the cam block outwardly away from the ramp surface of the femoral plate so as to move the femoral plate downwardly toward the tibial plate.

10. The orthopaedic surgical instrument assembly of claim 8, wherein:
the tibial plate has an anteroposteriorly-extending center line, and
a longitudinal axis of the drive screw is colinear with the center line of the tibial plate.

11. The orthopaedic surgical instrument assembly of claim 8, wherein the drive screw includes a socket defined in a proximal end thereof and a threaded shaft extending distally away from the socket.

12. A method of surgically preparing a patient's femur and tibia during an orthopaedic surgical knee procedure, comprising:
resecting a proximal end of the patient's tibia,
positioning the patient's femur and tibia in extension,
installing, with the patient's femur and tibia positioned in extension, a ligament balancer between the resected proximal end of the patient's tibia and an unresected pair of distal femoral condyles of the patient's femur,
securing, with the patient's femur and tibia positioned in extension, an extension pin guide to the installed ligament balancer,
installing a pair of guide pins into an anterior surface of the patient's femur with the extension pin guide,
removing the extension pin guide and installing a distal femoral cutting block onto the pair of guide pins installed on the anterior surface of the patient's femur,
performing a distal femoral resection on the patient's femur with the installed distal femoral cutting block so as to produce a planar resected distal femoral surface,
positioning the patient's femur and tibia in flexion,
installing, with the patient's femur and tibia positioned in flexion, the ligament balancer between the resected proximal end of the patient's tibia and an unresected pair of posterior femoral condyles of the patient's femur,
securing, with the patient's femur and tibia positioned in flexion, a flexion pin guide to the installed ligament balancer,
installing a pair of guide pins into the resected distal surface of the patient's femur with the flexion pin guide,
removing the flexion pin guide and installing a 4-in-1 femoral cutting block onto the pair of guide pins installed in the resected distal surface of the patient's femur, and
performing a number of additional femoral resections on the patient's femur with the installed 4-in-1 femoral cutting block.

13. The method of claim 12, further comprising operating, with the patient's femur and tibia positioned in extension, the installed ligament balancer to tension a number of knee ligaments of the patient prior to installation of the extension pin guide.

14. The method of claim 13, wherein operating the installed ligament balancer comprises operating a drive assembly of the ligament balancer to move a femoral plate of the ligament balancer away from a tibial plate of the ligament balancer.

15. The method of claim 14, wherein operating the drive assembly of the ligament balancer comprises urging a cam block into the femoral plate to move the femoral plate of the ligament balancer away from the tibial plate of the ligament balancer.

16. The method of claim 12, further comprising operating, with the patient's femur and tibia positioned in flexion, the installed ligament balancer to tension a number of knee ligaments of the patient prior to installation of the flexion pin guide.

17. The method of claim 16, wherein operating the installed ligament balancer comprises operating a drive assembly of the ligament balancer to move a femoral plate of the ligament balancer away from a tibial plate of the ligament balancer.

18. The method of claim 17, wherein operating the drive assembly of the ligament balancer comprises urging a cam block into the femoral plate to move the femoral plate of the ligament balancer away from the tibial plate of the ligament balancer.

19. The method of claim 12, wherein securing the extension pin guide to the installed ligament balancer comprises:
securing, with the patient's femur and tibia positioned in extension, a mounting block to the installed ligament balancer, and
securing the extension pin guide to the mounting block.

20. The method of claim 12, wherein securing the flexion pin guide to the installed ligament balancer comprises:
securing, with the patient's femur and tibia positioned in flexion, a mounting block to the installed ligament balancer, and
securing the flexion pin guide to the mounting block.
